(19) United States
(12) Patent Application Publication
(10) Pub. No.: US 2026/0305634 A1
(43) Pub. Date: Oct. 8, 2026

(54) CONCENTRATED MILK PROTEIN PREPARATIONS

(71) Applicant: 50G INC., Jamaica, NY (US)

(72) Inventor: Derek Netto, New York, NY (US)

(21) Appl. No.: 19/638,808

(22) Filed: Apr. 3, 2026

Related U.S. Application Data
(60) Provisional application No. 63/783,611, filed on Apr. 4, 2025.

(51) Int. Cl. A23C 9/142; A23C 9/14; A23C 9/152; A23J 3/08; A61K 35/20; A61K 38/17; B01D 61/02; B01D 61/14; B01D 61/58; B01D 69/02

(52) U.S. Cl. CPC A23C 9/1422; A23C 9/14; A23C 9/1427; A23C 9/1522; A23C 9/1528; A23J 3/08; A61K 35/20; A61K 38/1709; B01D 61/025; B01D 61/145; B01D 61/58; B01D 69/02; A23C 2210/206; B01D 2311/06; B01D 2315/16; B01D 2325/20

(57) ABSTRACT

Described herein are compositions of concentrated protein compositions derived from milk and processes for the preparation of said compositions.

### RELATED APPLICATIONS

This application claims the benefit of U.S. Provisional Patent Application No. 63/783,611, filed Apr. 4, 2025, the contents of which are fully incorporated by reference herein.

### BACKGROUND

Protein assists in muscle repair, immune function, and overall well-being, making it a cornerstone of dietary intake for athletes, aging populations, and individuals with medical conditions requiring specialized nutrition. Accordingly, concentrated protein solutions are needed to address global needs in nutrition and health.

Ultra-concentrated protein solutions enable the creation of compact, nutrient-dense products such as protein shakes, functional foods, and medical formulations, which are convenient, effective, and tailored to modern consumer lifestyles. Furthermore, as global protein demand rises with population growth, efficient production of high-quality protein products reduces resource use, waste, and environmental impact, supporting sustainability in food systems.

### SUMMARY OF THE INVENTION

In certain aspects provided herein is a liquid nutritional composition derived from milk, comprising:
protein at a protein concentration of at least about 0.15 g/mL;
optionally, fat at a fat concentration of less than about 0.01 g/mL; and
optionally, lactose, at a lactose concentration of less than about 0.01 g/mL.

In certain further aspects provided herein is a process for preparing a liquid nutritional composition comprising:
(i) filtering a first protein solution via ultrafiltration, thereby producing a second protein solution;
(ii) filtering the second protein solution via diafiltration, thereby producing a third protein solution; and
(iii) concentrating the third protein solution via one or more water-removal techniques, thereby producing the liquid nutritional composition.

Other features, objects, and advantages of the disclosure will be apparent from the detailed description, and from the claims.

### BRIEF DESCRIPTION OF THE DRAWINGS

FIG. 1 shows a schematic of the separation of milk into its components.

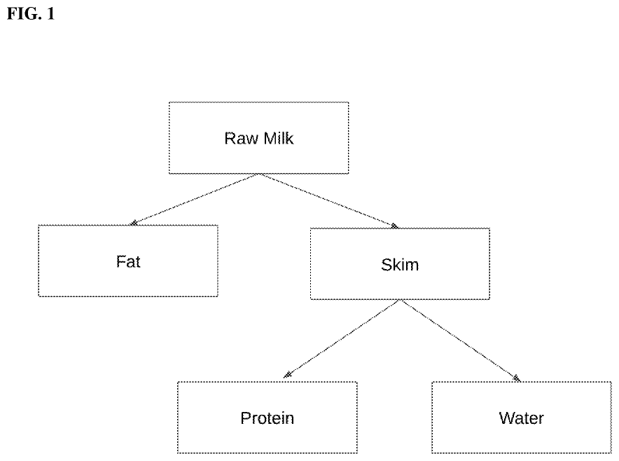

FIG. 2 is a schematic illustrating one embodiment of the process described herein.

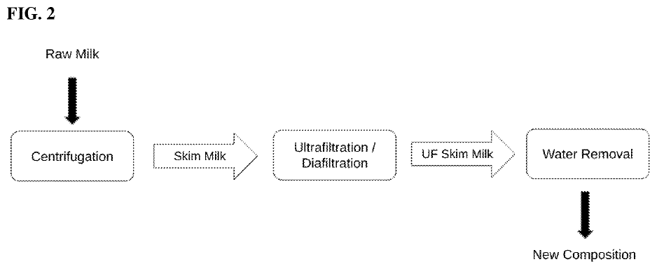

FIG. 3 is a simplified schematic illustrating one embodiment of the process described herein.

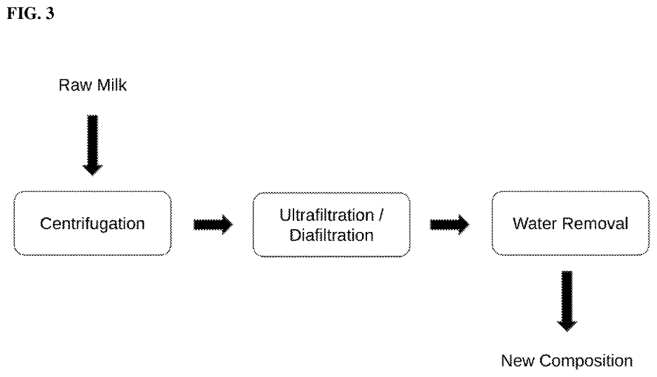

FIG. 4 is a schematic illustrating a traditional milk processing method.

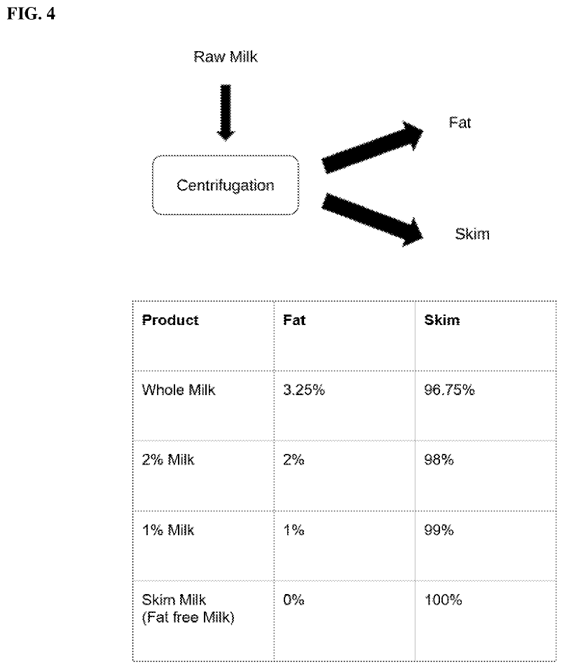

FIG. 5 is a schematic illustrating an ultrafiltered milk processing method.

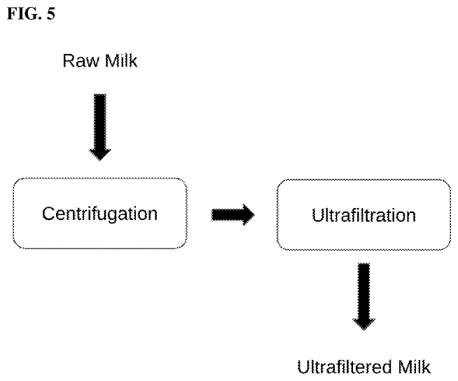

FIG. 6 is a schematic illustrating an evaporated milk processing method.

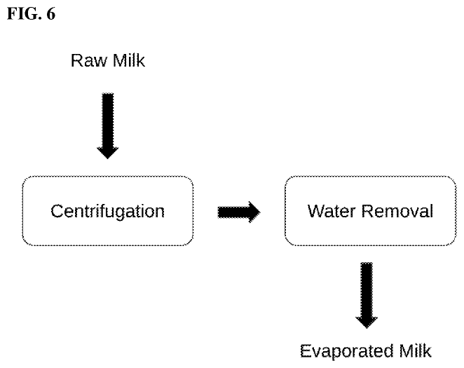

FIG. 7 shows a schematic of a process comprising ultrafiltration and reverse osmosis, resulting in a liquid nutritional composition comprises 23.25% protein.

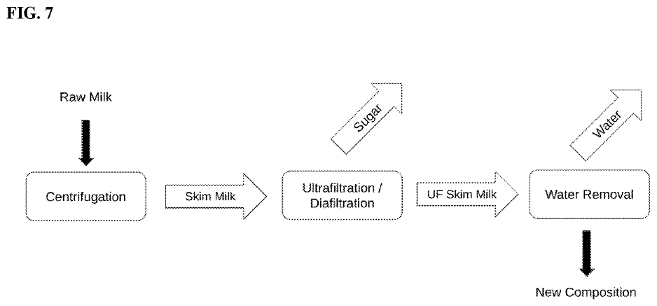

FIG. 8 shows a schematic of a process comprising ultrafiltration, resulting in a liquid nutritional composition comprises 7.28% protein.

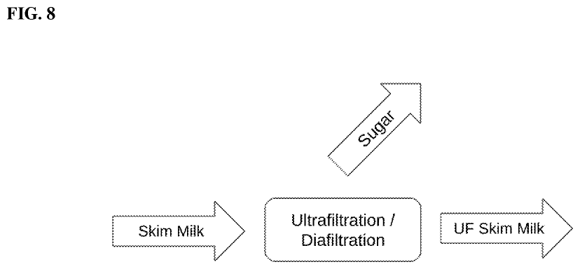

FIG. 9 shows a schematic of a process comprising reverse osmosis, resulting in a liquid nutritional composition comprises 12.28% protein.

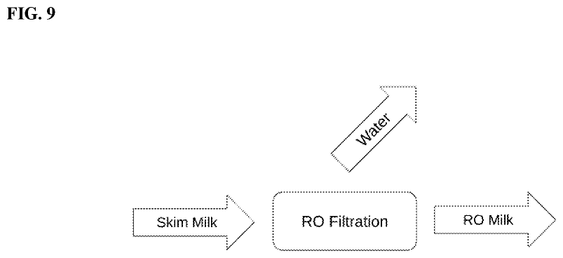

### DETAILED DESCRIPTION OF THE INVENTION

The present disclosure provides a process for producing hyper-concentrated dairy protein solutions by integrating ultrafiltration (UF), diafiltration, and reverse osmosis (RO). The process leverages the selective separation capability of UF and the water-removal efficiency of RO in a synergistic manner, with an optional intermediate optimization step to condition the retentate for maximum efficiency in the RO phase. The resulting compositions achieve protein concentrations exceeding 20.8% and are suitable for use as base ingredients in high-protein nutritional beverages, functional foods, and medical formulations.

Achieving ultra-high protein concentrations, such as 20.8% (~50 g protein in 240 mL), is a significant challenge with current ultrafiltration (UF) and reverse osmosis (RO) technologies. UF excels in selective separation but is limited by viscosity and membrane fouling at higher concentrations, while RO can remove water but struggles with overcoming osmotic pressure, membrane fouling, and lack of selectivity. These limitations lead to operational inefficiencies, increased costs, and inadequate scalability for commercial applications. Addressing these barriers is essential to meet the growing demand for ultra-concentrated protein solutions in nutritional and functional food markets.

### Compositions of the Invention

In certain aspects provided herein is a liquid nutritional composition derived from milk, comprising:
protein at a protein concentration of at least about 0.15 g/mL;
optionally, fat at a fat concentration of less than about 0.01 g/mL; and
optionally, lactose, at a lactose concentration of less than about 0.01 g/mL.

In certain embodiments, the milk is raw milk. In certain embodiments, the milk is skim milk.

In certain embodiments, the protein concentration is from about 0.15 to about 0.20 g/mL. In certain embodiments, the protein concentration is about 0.20 g/mL. In certain embodiments, the protein concentration is from about 0.20 to about 0.25 g/mL. In certain embodiments, the protein concentration is about 0.21 g/mL. In certain embodiments, the protein concentration is about 0.25 g/mL. In certain embodiments, the protein concentration is from about 0.25 to about 0.30 g/mL. In certain embodiments, the protein concentration is about 0.30 g/mL. In certain embodiments, the protein concentration is about 0.30 to about 0.35 g/mL. In certain embodiments, the protein concentration is about 0.35 g/mL. In certain embodiments, the protein concentration is about 0.35 to about 0.40 g/mL. In certain embodiments, the protein concentration is about 0.40 g/mL. In certain embodiments, the protein concentration is about 0.40 to about 0.45 g/mL. In certain embodiments, the protein concentration is about 0.45 g/mL. In certain embodiments, the protein concentration is about 0.45 to about 0.50 g/mL. In certain embodiments, the protein concentration is about 0.50 g/mL. In certain embodiments, the protein concentration is from about 0.15 g/mL to about 0.50 g/mL. In certain embodiments, the protein concentration is from about 0.20 g/mL to about 0.50 g/mL.

In certain embodiments, the protein concentration is ≥0.20 g/mL.

In certain embodiments, the protein concentration is at least about 36 grams per cup. In certain embodiments, the protein concentration is from about 36 grams per cup to about 40 grams per cup. In certain embodiments, the protein concentration is from about 40 grams per cup to about 45 grams per cup. In certain embodiments, the protein concentration is from about 45 grams per cup to about 50 grams per cup. In certain embodiments, the protein concentration is about 48 grams per cup. In certain embodiments, the protein concentration is from about 50 grams per cup to about 55 grams per cup. In certain embodiments, the protein concentration is about 50 grams per cup. In certain embodiments, the protein concentration is from about 55 grams per cup to about 60 grams per cup. In certain embodiments, the protein concentration is about 60 grams per cup. In certain embodiments, the protein concentration is about 60 grams per cup to about 65 grams per cup. In certain embodiments, the protein concentration is from about 65 grams per cup to about 70 grams per cup. In certain embodiments, the protein concentration is from about 70 grams per cup to about 75 grams per cup. In certain embodiments, the protein concentration is about 72 grams per cup. In certain embodiments, the protein concentration is from about 75 grams per cup to about 80 grams per cup. In certain embodiments, the protein concentration is from about 80 to about 85 grams per cup. In certain embodiments, the protein concentration is about 84 grams per cup. In certain embodiments, the protein concentration is from about 85 to about 90 grams per cup. In certain embodiments, the protein concentration is from about 90 to about 95 grams per cup. In certain embodiments, the protein concentration is from about 95 to about 100 grams per cup. In certain embodiments, the protein concentration is from about 100 to about 105 grams per cup. In certain embodiments, the protein concentration is from about 105 to about 110 grams per cup. In certain embodiments, the protein concentration is from about 110 to about 115 grams per cup. In certain embodiments, the protein concentration is from about 115 to about 120 grams per cup. In certain embodiments, the protein concentration is from about 36 grams per cup to about 120 grams per cup.

In certain embodiments, the protein concentration is ≥40 grams per cup.

In certain embodiments, the liquid nutritional composition comprises between about 15% (w/v) protein to about 20% (w/v) protein. In certain embodiments, the liquid nutritional composition comprises between about 20% (w/v) protein to about 25% (w/v) protein. In certain embodiments, the liquid nutritional composition comprises between about 25% (w/v) protein to about 30% (w/v) protein. In certain embodiments, the liquid nutritional composition comprises between about 30% (w/v) protein to about 35% (w/v) protein. In certain embodiments, the liquid nutritional composition comprises between about 35% (w/v) protein to about 40% (w/v) protein. In certain embodiments, the liquid nutritional composition comprises between about 40% (w/v) protein to about 45% (w/v) protein. In certain embodiments, the liquid nutritional composition comprises between about 15% (w/v) protein to about 45% (w/v) protein.

In certain embodiments, the fat concentration is about 0 g/mL.

In certain embodiments, the lactose concentration is about 0 g/mL.

In certain embodiments, the composition has a viscosity between about 1 mPa-s to about 1000 mPa-s. In certain embodiments, the composition has viscosity of about 100 mPa-s. In certain preferred embodiments, the composition has viscosity of less than about 100 mPa-s. In certain embodiments, the composition has a viscosity between about 1000 mPa-s to about 2000 mPa-s. In certain embodiments, the composition has a viscosity between about 2000 mPa-s to about 3000 mPa-s. In certain embodiments, the composition has a viscosity between about 3000 mPa-s to about 4000 mPa-s. In certain embodiments, the composition has a viscosity between about 4000 mPa-s to about 5000 mPa-s. In certain embodiments, the composition has a viscosity between about 1 mPa-s to about 5000 mPa-s.

In certain embodiments, the composition is a shelf-stable composition. In certain such embodiments, the shelf-stable composition is packaged in Ultra-High Temperature (UHT) packaging. In other such embodiments, the shelf-stable composition is packaged in aseptic packaging. In certain embodiments, the shelf-stable composition comprises in-container retort processing (e.g., sterilization while sealed in the package).

In certain embodiments, the composition is prepared in a serving size of less than about 120 ml. In certain embodiments, the composition is prepared in a serving size of less than about 100 ml. In certain embodiments, the composition is prepared in a serving size of less than about 80 ml. In certain embodiments, the composition is prepared in a serving size of less than about 60 ml. In certain embodiments, the composition is prepared in a serving size of less than about 40 ml. In certain embodiments, the composition is prepared in a serving size of less than about 20 ml.

In certain embodiments, the composition is prepared in a serving size of less than about 4 ounces. In certain embodiments, the composition is prepared in a serving size of less than about 3.5 ounces. In certain embodiments, the composition is prepared in a serving size of less than about 3 ounces. In certain embodiments, the composition is prepared in a serving size of less than about 2.5 ounces. In certain embodiments, the composition is prepared in a serving size of less than about 2 ounces. In certain embodiments, the composition is prepared in a serving size of less than about 1.5 ounces. In certain embodiments, the composition is prepared in a serving size of less than about 1 ounce.

### Processes of the Invention

In certain aspects provided herein is a process for preparing a liquid nutritional composition comprising:
(i) filtering a first protein solution via ultrafiltration, thereby producing a second protein solution;
(ii) filtering the second protein solution via diafiltration, thereby producing a third protein solution; and
(iii) concentrating the third protein solution via one or more water-removal techniques, thereby producing the liquid nutritional composition.

In certain embodiments, the first protein solution is derived from milk. In certain embodiments, the first protein solution is derived from skim milk. In certain embodiments, the first protein solution is derived from raw milk. In certain such embodiments, the first protein solution is prepared by centrifugation of raw milk.

In certain embodiments, the first protein solution is derived from whey.

In certain embodiments, the ultrafiltration employs a filtration membrane comprising a molecular weight cut-off of about 10,000 Da.

In certain embodiments, the ultrafiltration is carried out at a suitable ultrafiltration temperature, and the suitable ultrafiltration temperature is from about 40 to about 50° F. In certain embodiments, the ultrafiltration is carried out at a suitable ultrafiltration temperature, and the suitable ultrafiltration temperature is from about 50 to about 60° F. In certain embodiments, the ultrafiltration is carried out at a suitable ultrafiltration temperature, and the suitable ultrafiltration temperature is from about 60 to about 70° F. In certain embodiments, the ultrafiltration is carried out at a suitable ultrafiltration temperature, and the suitable ultrafiltration temperature is from about 70 to about 80° F. In certain embodiments, the ultrafiltration is carried out at a suitable ultrafiltration temperature, and the suitable ultrafiltration temperature is from about 80 to about 90° F. In certain embodiments, the ultrafiltration is carried out at a suitable ultrafiltration temperature, and the suitable ultrafiltration temperature is from about 90 to about 100° F. In certain embodiments, the ultrafiltration is carried out at a suitable ultrafiltration temperature, and the suitable ultrafiltration temperature is from about 100 to about 110° F. In certain embodiments, the ultrafiltration is carried out at a suitable ultrafiltration temperature, and the suitable ultrafiltration temperature is from about 40 to about 110° F.

In certain embodiments, the ultrafiltration is carried out at a suitable ultrafiltration pressure, and the suitable ultrafiltration pressure is from about 50 psi to about 60 psi. In certain embodiments, the ultrafiltration is carried out at a suitable ultrafiltration pressure, and the suitable ultrafiltration pressure is from about 60 psi to about 70 psi. In certain embodiments, the ultrafiltration is carried out at a suitable ultrafiltration pressure, and the suitable ultrafiltration pressure is from about 70 psi to about 80 psi. In certain embodiments, the ultrafiltration is carried out at a suitable ultrafiltration pressure, and the suitable ultrafiltration pressure is from about 80 psi to about 90 psi. In certain embodiments, the ultrafiltration is carried out at a suitable ultrafiltration pressure, and the suitable ultrafiltration pressure is from about 90 psi to about 100 psi. In certain embodiments, the ultrafiltration is carried out at a suitable ultrafiltration pressure, and the suitable ultrafiltration pressure is from about 100 psi to about 110 psi. In certain embodiments, the ultrafiltration is carried out at a suitable ultrafiltration pressure, and the suitable ultrafiltration pressure is from about 110 psi to about 120 psi. In certain embodiments, the ultrafiltration is carried out at a suitable ultrafiltration pressure, and the suitable ultrafiltration pressure is from about 120 psi to about 130 psi. In certain embodiments, the ultrafiltration is carried out at a suitable ultrafiltration pressure, and the suitable ultrafiltration pressure is from about 130 psi to about 140 psi. In certain embodiments, the ultrafiltration is carried out at a suitable ultrafiltration pressure, and the suitable ultrafiltration pressure is from about 140 psi to about 150 psi. In certain embodiments, the ultrafiltration is carried out at a suitable ultrafiltration pressure, and the suitable ultrafiltration pressure is from about 50 psi to about 150 psi.

In certain embodiments, diafiltration comprises adding a suitable amount of water to the second protein solution. In certain such embodiments, the added water is filtered through the filtration membrane.

In certain embodiments, the diafiltration is carried out at a suitable diafiltration temperature, and the suitable diafiltration temperature is from about 40 to about 50° F. In certain embodiments, the diafiltration is carried out at a suitable diafiltration temperature, and the suitable diafiltration temperature is from about 50 to about 60° F. In certain embodiments, the diafiltration is carried out at a suitable diafiltration temperature, and the suitable diafiltration temperature is from about 60 to about 70° F. In certain embodiments, the diafiltration is carried out at a suitable diafiltration temperature, and the suitable diafiltration temperature is from about 70 to about 80° F. In certain embodiments, the diafiltration is carried out at a suitable diafiltration temperature, and the suitable diafiltration temperature is from about 80 to about 90° F. In certain embodiments, the diafiltration is carried out at a suitable diafiltration temperature, and the suitable diafiltration temperature is from about 90 to about 100° F. In certain embodiments, the diafiltration is carried out at a suitable diafiltration temperature, and the suitable diafiltration temperature is from about 100 to about 110° F. In certain embodiments, the diafiltration is carried out at a suitable diafiltration temperature, and the suitable diafiltration temperature is from about 40 to about 110° F.

In certain embodiments, the diafiltration is carried out at a suitable diafiltration pressure, and the suitable diafiltration pressure is from about 50 psi to about 60 psi. In certain embodiments, the diafiltration is carried out at a suitable diafiltration pressure, and the suitable diafiltration pressure is from about 60 psi to about 70 psi. In certain embodiments, the diafiltration is carried out at a suitable diafiltration pressure, and the suitable diafiltration pressure is from about 70 psi to about 80 psi. In certain embodiments, the diafiltration is carried out at a suitable diafiltration pressure, and the suitable diafiltration pressure is from about 80 psi to about 90 psi. In certain embodiments, the diafiltration is carried out at a suitable diafiltration pressure, and the suitable diafiltration pressure is from about 90 psi to about 100 psi. In certain embodiments, the diafiltration is carried out at a suitable diafiltration pressure, and the suitable diafiltration pressure is from about 100 psi to about 110 psi. In certain embodiments, the diafiltration is carried out at a suitable diafiltration pressure, and the suitable diafiltration pressure is from about 110 psi to about 120 psi. In certain embodiments, the diafiltration is carried out at a suitable diafiltration pressure, and the suitable diafiltration pressure is from about 120 psi to about 130 psi. In certain embodiments, the diafiltration is carried out at a suitable diafiltration pressure, and the suitable diafiltration pressure is from about 130 psi to about 140 psi. In certain embodiments, the diafiltration is carried out at a suitable diafiltration pressure, and the suitable diafiltration pressure is from about 140 psi to about 150 psi. In certain embodiments, the diafiltration is carried out at a suitable diafiltration pressure, and the suitable diafiltration pressure is from about 50 psi to about 150 psi. In certain embodiments, the diafiltration is carried out at a suitable diafiltration pressure, and the suitable diafiltration pressure is from about 50 psi to about 150 psi.

In certain embodiments, the one or more water-removal techniques is reverse osmosis. In certain embodiments, the reverse osmosis employs a membrane comprising a molecular weight cut-off from about 50 Da to about 60 Da. In certain embodiments, the reverse osmosis employs a membrane comprising a molecular weight cut-off from about 60 Da to about 70 Da. In certain embodiments, the reverse osmosis employs a membrane comprising a molecular weight cut-off from about 70 Da to about 80 Da. In certain embodiments, the reverse osmosis employs a membrane comprising a molecular weight cut-off from about 50 Da to about 80 Da.

In certain embodiments, the one or more water-removal technique is distillation.

In certain embodiments, the one or more water-removal techniques are carried out at a suitable water-removal temperature, and the suitable water-removal temperature is from about 40 to about 50° F. In certain embodiments, the water-removal is carried out at a suitable water-removal temperature, and the suitable water-removal temperature is from about 50 to about 60° F. In certain embodiments, the water-removal is carried out at a suitable water-removal temperature, and the suitable water-removal temperature is from about 60 to about 70° F. In certain embodiments, the water-removal is carried out at a suitable water-removal temperature, and the suitable water-removal temperature is from about 70 to about 80° F. In certain embodiments, the water-removal is carried out at a suitable water-removal temperature, and the suitable water-removal temperature is from about 80 to about 90° F. In certain embodiments, the water-removal is carried out at a suitable water-removal temperature, and the suitable water-removal temperature is from about 90 to about 100° F. In certain embodiments, the water-removal is carried out at a suitable water-removal temperature, and the suitable water-removal temperature is from about 100 to about 110° F. In certain embodiments, the water-removal is carried out at a suitable water-removal temperature, and the suitable water-removal temperature is from about 40 to about 110° F.

In certain embodiments, the one or more water-removal techniques are carried out at a suitable water-removal pressure, and the suitable water-removal pressure is from about 400 psi to about 450 psi. In certain embodiments, the one or more water-removal techniques are carried out at a suitable water-removal pressure, and the suitable water-removal pressure is from about 450 psi to about 500 psi. In certain embodiments, the one or more water-removal techniques are carried out at a suitable water-removal pressure, and the suitable water-removal pressure is from about 500 psi to about 550 psi. In certain embodiments, the one or more water-removal techniques are carried out at a suitable water-removal pressure, and the suitable water-removal pressure is from about 550 psi to about 600 psi. In certain embodiments, the one or more water-removal techniques are carried out at a suitable water-removal pressure, and the suitable water-removal pressure is from about 400 psi to about 600 psi.

In certain embodiments, the process provided herein further comprising adjusting the pH of the third protein solution to between about 6.0 to about 7.5 prior to concentrating the third protein solution. In certain embodiments, the process provided herein further comprising adjusting the pH of the third protein solution to between about 6.0 to about 6.5 prior to concentrating the third protein solution. In certain embodiments, the process provided herein further comprising adjusting the pH of the third protein solution to between about 6.5 to about 7.0 prior to concentrating the third protein solution. In certain embodiments, the process provided herein further comprising adjusting the pH of the third protein solution to between about 7.0 to about 7.5 prior to concentrating the third protein solution.

In certain embodiments, the process provided herein further comprising adjusting the temperature of the third protein solution to between about 35 to about 50° C. prior to concentrating the third protein solution. In certain embodiments, the process provided herein further comprising adjusting the temperature of the third protein solution to between about 35 to about 40° C. prior to concentrating the third protein solution. In certain embodiments, the process provided herein further comprising adjusting the temperature of the third protein solution to between about 40 to about 45° C. prior to concentrating the third protein solution. In certain embodiments, the process provided herein further comprising adjusting the temperature of the third protein solution to between about 45 to about 50° C. prior to concentrating the third protein solution.

In certain embodiments, the process provided herein further comprising adding between about 1 mM to about 150 mM of one or more salts to the third protein solution prior to concentrating the third protein solution, wherein the one or more salts are selected from calcium chloride, sodium chloride, potassium chloride, sodium citrate, disodium phosphate, magnesium chloride, calcium lactate, or combinations thereof. In certain embodiments, the process provided herein further comprising adding from about 1 mM to about 50 mM of the one or more salts to the third protein solution prior to concentrating the third protein solution. In certain embodiments, the process provided herein further comprising adding from about 50 mM to about 100 mM of the one or more salts to the third protein solution prior to concentrating the third protein solution. In certain embodiments, the process provided herein further comprising adding from about 100 mM to about 150 mM of the one or more salts to the third protein solution prior to concentrating the third protein solution.

In certain embodiments, the process provided herein further comprising adding lecithin or polysorbates to the third protein solution prior to concentrating the third protein solution. In certain such embodiments, the process provided herein further comprising adding lecithin to the third protein solution prior to concentrating the third protein solution. In certain embodiments, the process provided herein further comprising adding polysorbates to the third protein solution prior to concentrating the third protein solution.

In certain embodiments, the process provided herein further comprising homogenization of the third protein solution at a homogenization pressure of between about 50 to about 150 bar prior to concentrating the third protein solution. In certain embodiments, the process provided herein further comprising homogenization of the third protein solution at a homogenization pressure of between about 50 to about 100 bar prior to concentrating the third protein solution. In certain embodiments, the process provided herein further comprising homogenization of the third protein solution at a homogenization pressure of between about 100 to about 150 bar prior to concentrating the third protein solution.

In certain embodiments, the process for preparing a liquid nutritional composition does not comprise step (i). In certain embodiments, the process for preparing a liquid nutritional composition does not comprise step (ii). In certain embodiments, the process for preparing a liquid nutritional composition does not comprise step (iii). In certain embodiments, the process for preparing a liquid nutritional composition comprises steps (i)-(iii) in any order.

In certain embodiments, raw milk undergoes centrifugation, thereby producing the first protein solution.

In certain embodiments, the protein concentration is from about 0.15 to about 0.20 g/mL. In certain embodiments, the protein concentration is about 0.20 g/mL. In certain embodiments, the protein concentration is from about 0.20 to about 0.25 g/mL. In certain embodiments, the protein concentration is about 0.21 g/mL. In certain embodiments, the protein concentration is about 0.25 g/mL. In certain embodiments, the protein concentration is from about 0.25 to about 0.30 g/mL. In certain embodiments, the protein concentration is about 0.30 g/mL. In certain embodiments, the protein concentration is about 0.30 to about 0.35 g/mL. In certain embodiments, the protein concentration is about 0.35 g/mL. In certain embodiments, the protein concentration is about 0.35 to about 0.40 g/mL. In certain embodiments, the protein concentration is about 0.40 g/mL. In certain embodiments, the protein concentration is about 0.40 to about 0.45 g/mL. In certain embodiments, the protein concentration is about 0.45 g/mL. In certain embodiments, the protein concentration is about 0.45 to about 0.50 g/mL. In certain embodiments, the protein concentration is about 0.50 g/mL. In certain embodiments, the protein concentration is from about 0.15 g/mL to about 0.50 g/mL.

In certain embodiments, the protein concentration is at least about 36 grams per cup. In certain embodiments, the protein concentration is from about 36 grams per cup to about 40 grams per cup. In certain embodiments, the protein concentration is from about 40 grams per cup to about 45 grams per cup. In certain embodiments, the protein concentration is from about 45 grams per cup to about 50 grams per cup. In certain embodiments, the protein concentration is about 48 grams per cup. In certain embodiments, the protein concentration is from about 50 grams per cup to about 55 grams per cup. In certain embodiments, the protein concentration is about 50 grams per cup. In certain embodiments, the protein concentration is from about 55 grams per cup to about 60 grams per cup. In certain embodiments, the protein concentration is about 60 grams per cup. In certain embodiments, the protein concentration is about 60 grams per cup to about 65 grams per cup. In certain embodiments, the protein concentration is from about 65 grams per cup to about 70 grams per cup. In certain embodiments, the protein concentration is from about 70 grams per cup to about 75 grams per cup. In certain embodiments, the protein concentration is about 72 grams per cup. In certain embodiments, the protein concentration is from about 75 grams per cup to about 80 grams per cup. In certain embodiments, the protein concentration is from about 80 to about 85 grams per cup. In certain embodiments, the protein concentration is about 84 grams per cup. In certain embodiments, the protein concentration is from about 85 to about 90 grams per cup. In certain embodiments, the protein concentration is from about 90 to about 95 grams per cup. In certain embodiments, the protein concentration is from about 95 to about 100 grams per cup. In certain embodiments, the protein concentration is from about 100 to about 105 grams per cup. In certain embodiments, the protein concentration is from about 105 to about 110 grams per cup. In certain embodiments, the protein concentration is from about 110 to about 115 grams per cup. In certain embodiments, the protein concentration is from about 115 to about 120 grams per cup. In certain embodiments, the protein concentration is from about 36 grams per cup to about 120 grams per cup.

In certain embodiments, the liquid nutritional composition comprises between about 15% (w/v) protein to about 20% (w/v) protein. In certain embodiments, the liquid nutritional composition comprises between about 20% (w/v) protein to about 25% (w/v) protein. In certain embodiments, the liquid nutritional composition comprises between about 25% (w/v) protein to about 30% (w/v) protein. In certain embodiments, the liquid nutritional composition comprises between about 30% (w/v) protein to about 35% (w/v) protein. In certain embodiments, the liquid nutritional composition comprises between about 35% (w/v) protein to about 40% (w/v) protein. In certain embodiments, the liquid nutritional composition comprises between about 40% (w/v) protein to about 45% (w/v) protein. In certain embodiments, the liquid nutritional composition comprises between about 15% (w/v) protein to about 45% (w/v) protein.

In certain embodiments, if the liquid nutritional composition comprises fat, the fat is present at a concentration of less than about 0.01 g/mL.

In certain embodiments, if the liquid nutritional composition comprises lactose, the lactose is present at a concentration of less than about 0.01 g/mL.

In certain embodiments, the composition has a viscosity between about 1 mPa-s to about 1000 mPa-s. In certain embodiments, the composition has viscosity of about 100 mPa-s. In certain preferred embodiments, the composition has viscosity of less than about 100 mPa-s. In certain embodiments, the composition has a viscosity between about 1000 mPa-s to about 2000 mPa-s. In certain embodiments, the composition has a viscosity between about 2000 mPa-s to about 3000 mPa-s. In certain embodiments, the composition has a viscosity between about 3000 mPa-s to about 4000 mPa-s. In certain embodiments, the composition has a viscosity between about 4000 mPa-s to about 5000 mPa-s. In certain embodiments, the composition has a viscosity between about 1 mPa-s to about 5000 mPa-s.

In certain embodiments, a liquid nutritional composition produced by the process described herein.

### Limitations of Existing Processes

Limitations of UF alone-UF effectively separates proteins from smaller solutes like lactose and minerals but is fundamentally constrained by viscosity and membrane fouling as protein concentration increases. These issues reduce membrane efficiency, prolong processing times, and prevent UF from achieving protein concentrations beyond 5-12% solids. This limitation renders UF unsuitable for applications requiring ultra-high protein densities.

Limitations of RO alone-RO removes water from liquid streams but lacks the selectivity needed for protein isolation. At higher concentrations, osmotic pressure rises exponentially, making further water removal impractical or requiring prohibitively high operating pressures. This not only limits achievable protein concentrations but also increases energy demands. Additionally, membrane fouling and increased viscosity exacerbate operational inefficiencies, further reducing RO's effectiveness for ultra-high-protein applications.

Fouling and Maintenance-Both UF and RO membranes are prone to fouling due to protein aggregation and scaling, especially when processing high-protein solutions. Frequent cleaning cycles lead to increased downtime, higher maintenance costs, and reduced operational efficiency. These challenges are particularly pronounced in commercial-scale operations where high throughput is required.

Energy Inefficiency—The energy consumption of UF and RO increases dramatically with higher protein concentrations. RO, in particular, becomes economically and technically inefficient due to the high pressures required to overcome osmotic pressure. This energy intensity makes standalone RO or UF unsuitable for cost-effective, high-volume production of ultra-concentrated protein solutions.

### Unmet Needs

Productivity and Yield—Standalone UF and RO systems are unable to meet the productivity and yield requirements for large-scale production of ultra-concentrated proteins. Achieving concentrations beyond 10-12% solids often requires excessive processing times and energy inputs, leading to diminished returns in high-volume commercial applications.

Efficiency—Energy consumption, frequent membrane replacement, and fouling-related downtime associated with UF and RO may make it difficult to efficiently produce ultra-concentrated protein solutions.

Despite the above, there is a rapidly growing need for ultra-concentrated protein solutions in nutritional beverages, functional foods, and medical nutrition. Protein bases are needed that exhibit high purity, precise macronutrient profiles, and minimal impurities. The technological limitations of UF and RO prevent these systems from adequately meeting these needs.

To overcome these technological disadvantages, the present disclosure provides an integrated approach that combines the selective separation capabilities of UF with the water-removal efficiency of RO. By addressing viscosity, fouling, osmotic pressure, and energy inefficiency, the disclosed processes enable the scalable and cost-effective production of ultra-concentrated protein solutions, unlocking new opportunities in the growing nutritional and functional food markets.

The process described herein combines the selective separation capabilities of UF with the water-removal efficiency of RO, addressing the limitations of each method when used independently. In certain embodiments, optional intermediate optimization steps may further enhance the system by conditioning the UF retentate to improve compatibility with RO membranes, ensuring efficient water removal and minimizing fouling.

In certain embodiments, the process described herein begins with ultrafiltration, where milk or whey is passed through a semi-permeable membrane with a molecular weight cutoff (MWCO) of 10,000 Daltons. This membrane selectively retains high-molecular-weight proteins, such as casein micelles and whey proteins, while allowing smaller molecules like lactose, minerals, and water to permeate.

Operating at a transmembrane pressure (TMP) of 150 psi, with feed temperatures maintained at 40-110° F., the UF phase enriches the protein concentration to 5-12% solids. The removal of smaller solutes reduces osmotic pressure and viscosity in the retentate, enabling more efficient downstream processing.

In certain embodiments, the process comprises one or more intermediate steps, wherein the UF retentate undergoes an intermediate conditioning step to enhance its compatibility with the RO phase. This step may involve:
1. pH Adjustment: Stabilizing proteins by adjusting the pH to 6.0-7.5.
2. Temperature Control: Reducing viscosity by maintaining temperatures of 35-50° C.
3. Ionic Strength Adjustment: Adding salts such as calcium chloride, e.g. at 10-50 mM to stabilize protein structures.
4. Anti-Fouling Agents: Introducing lecithin or polysorbates to prevent membrane fouling.
5. Homogenization: Ensuring uniform particle size distribution through high-pressure homogenization at 50-150 bar.

These optimizations may help the retentate maintains flow properties and prevents fouling during high-pressure filtration in the RO phase.

The conditioned retentate is passed through reverse osmosis membranes with pore sizes smaller than 0.5 nanometers. These membranes remove water while retaining proteins and other high-molecular-weight components. The UF pre-concentration reduces osmotic pressure, allowing the RO phase to operate efficiently at 600 psi. The RO system achieves protein concentrations exceeding 20.8%, which may be suitable for advanced nutritional and functional food applications.

### Overcoming Limitations of UF and RO

Conventional ultrafiltration (UF) and reverse osmosis (RO) processes are constrained by their inherent limitations when used independently. UF systems typically cap at 5-12% protein concentration due to viscosity-induced flow restrictions and fouling caused by protein aggregation on the membrane surface. Conversely, RO systems struggle to exceed 12-15% solids, as osmotic pressure increases exponentially at higher concentrations, requiring impractically high pressures to remove additional water. Additionally, RO membranes do not selectively separate proteins, resulting in a mixture of concentrated solutes. The process described herein integrates the strengths of both methods (Table 1), where UF selectively separates proteins from smaller solutes, and RO efficiently removes water, to achieve protein concentrations exceeding 20.8% solids (~50 g protein in 240 mL). This integrated approach allows for a protein concentrations that neither method can achieve independently. Protein concentration processing using the process described herein may achieve a protein concentration of 23.25 g/100 g

TABLE 1
Processing Methods of Existing Products
vs. Processing Methods Described herein

| | | Water |
|---|---|---|
| Name | Ultrafiltration | Removal |
| Generic Whole Milk | No | No |
| Generic 2% Milk | No | No |
| Generic 1% Milk | No | No |
| Generic Skim Milk | No | No |
| Fairlife UF Whole Milk | Yes | No |
| Fairlife UF 2% Milk | Yes | No |
| Fairlife UF 1% Milk | Yes | No |
| Fairlife UF Skim Milk | Yes | No |
| Slate Milk | Yes | No |
| Fairlife Core Power Shake | Yes | No |
| Nestle Evaporated Milk | No | Yes |
| Nestle Condensed Milk | No | Yes |
| Fairlife Nutrition Plan Shake | Yes | No |
| Fairlife Core Power Elite Shake | Yes | No |
| O-AT-KA Ultrafiltered (UF) Skim | Yes | No |
| Composition described herein | Yes | Yes |

Reduction in Osmotic Pressure-One aspect of the process described herein is the pre-removal of smaller solutes such as lactose and minerals during the UF phase, which may significantly reduce the osmotic pressure in the RO phase. Osmotic pressure may be a major limiting factor in traditional RO systems, as the energy required for water removal increases exponentially with solute concentration. By eliminating a substantial portion of low-molecular-weight solutes before the RO phase, the process described herein may enable the RO system to operate at lower pressures (typically 400 psi) while maintaining high water flux. This not only reduces energy consumption but also lowers the mechanical stress on the RO membranes, extending their lifespan and improving long-term operational efficiency.

Mitigation of Fouling-Fouling may be a challenge in both UF and RO systems, leading to reduced membrane performance, increased downtime, and higher maintenance costs. The optional intermediate optimization step in the process described herein may be specifically designed to address fouling. By stabilizing proteins through pH adjustment, ionic strength optimization, and temperature control, this step may reduce protein aggregation and minimizes the formation of a fouling layer on the membrane surface. Furthermore, the addition of food-grade anti-fouling agents (e.g., lecithin or polysorbates) and high-pressure homogenization may ensure uniform particle size distribution, reducing localized viscosity and membrane blockages. These optional steps may enhance operational efficiency, decrease the frequency of cleaning cycles, and significantly prolong the lifespan of UF and RO membranes.

Energy Efficiency—Energy efficiency may be achieved by addressing the root causes of energy-intensive water removal in RO systems. In certain embodiments, while the total liquid volume entering the RO phase may not be reduced, the UF phase may selectively removes dissolved solutes, decreasing osmotic pressure and enabling the RO system to achieve the desired level of water removal at lower pressures. By optimizing membrane compatibility and flow properties through intermediate conditioning, the system may further minimize resistance caused by high viscosity. These combined steps may allow the RO phase to operate with greater energy efficiency compared to traditional standalone RO processes. The reduction in energy demand may make the integrated UF-RO process both cost-effective and environmentally sustainable and may provide an advantage in high-volume industrial applications.

Scalability—The modular design of the integrated UF-RO system may enable seamless scaling for industrial production. Each step-UF, intermediate optimization, and RO—may be independently adjusted to accommodate varying feed volumes and product specifications and may provide flexibility for different manufacturing scales. This adaptability may make the process described herein suitable for a wide range of applications, from small-scale specialty formulations to large-scale production of ultra-concentrated protein solutions. Additionally, the robust design and streamlined workflow of the described process may reduce operational complexity, and may ensure reliable and efficient performance at high production volumes. This scalability may align with the growing market demand for high-protein products, and may enable manufacturers to efficiently meet consumer needs in the nutritional and functional food sectors.

Conventional ultrafiltration (UF) and reverse osmosis (RO) processes are used in dairy and protein production but may face significant limitations when used independently. UF systems may be constrained by viscosity and fouling at higher protein concentrations, capping their effectiveness at 5-12% solids. RO systems, while effective at water removal, may struggle with osmotic pressure and fouling at high concentrations and lack the ability to selectively separate proteins from other solutes. These limitations may prevent either method from achieving the ultra-high protein concentrations required for advanced nutritional applications.

The process described herein integrates UF and RO in a sequential and optimized process and may overcome the limitations of traditional processes. The use of a 10,000 MWCO UF membrane to selectively remove smaller solutes, coupled with an optional intermediate conditioning step to stabilize the retentate for RO, may enable the production of protein concentrations exceeding 20.8% solids (~50 g protein in 240 mL). Traditional Processes do not utilize a combined UF-RO system with optional intermediate optimization designed to address challenges such as viscosity, fouling, and osmotic pressure. By leveraging the strengths of both methods and addressing their weaknesses, the methods and compositions described herein may establish an efficient and scalable approach to producing ultra-high protein concentrates.

The ultra-high protein concentrates produced by the process described herein may be essential for developing nutritional beverages, functional foods, and medical formulations. By meeting the growing demand for nutrient-dense, compact products, the process and composition described herein may support advancements in health and nutrition. Additionally, the described process may contribute to sustainable food production by improving resource efficiency, reducing waste, and lowering energy consumption.

### Exemplary Benefits and Applications of Compositions and Methods Described Herein

The following benefits which are included merely for purposes of illustration of certain aspects and embodiments of the present disclosure, and are not intended to limit the methods and compositions described herein.

Increased Protein Content—The composition described herein has a significantly higher protein content compared to regular milk, making it an excellent source of high-quality protein.

High-Protein Shakes—May be used as a base or an additive to enhance the protein content in shakes, making them suitable for athletes, bodybuilders, and individuals seeking to increase their protein intake.

Ice Cream—May be incorporated into ice cream formulations to create high-protein ice cream, offering a nutritious and appealing dessert option.

Yogurt—May be used in yogurt production to boost protein content, resulting in a more nutritious product that appeals to health-conscious consumers.

Baked Goods—May be added to recipes for bread, muffins, and other baked goods to enhance their protein content.

Smoothies and Beverages—May be used in smoothies and other beverages to provide an extra protein boost.

Muscle Maintenance and Growth—May support muscle maintenance and growth, making it ideal for athletes and individuals engaged in regular physical activity.

Weight Management—High-protein foods may help increase feelings of fullness and satiety, aiding in weight management.

Metabolism—May help boost metabolism due the higher thermic effect of proteins compared to fats and carbohydrates.

Consumer Trends—There is a growing demand for high-protein foods and beverages among consumers, driven by health and wellness trends.

Dietary Needs—May be suitable for various dietary preferences, including low-carb, ketogenic, and high-protein diets.

Texture and Consistency—The composition described herein may improve the texture and consistency of food products, enhancing their mouthfeel and overall sensory experience.

Value Addition—By incorporating the composition described herein, manufacturers may create value-added products that can be marketed at a premium price.

Product Differentiation—May allow manufacturers to differentiate their products in a competitive market by offering unique, high-protein options.

Easy Integration—The compositions of the present disclosure may be easily integrated into various manufacturing processes, saving time and effort in product development.

### Definitions

Unless otherwise defined, all technical and scientific terms used herein have the same meaning as commonly understood by one of ordinary skill in the art to which this methods and compositions of the present disclosure belong. Although methods and materials similar or equivalent to those described herein can be used in the practice or testing of the methods and compositions described herein, suitable methods and materials are described below.

As used herein, the term “ultrafiltration” refers to a type of filtration, driven by a pressure gradient, which typically has a molecular weight cutoff (MWCO) range from about 3,000 to about 100,000 Da. In milk separation/fractionation processes, ultrafiltration is traditionally used for fractionation of whey proteins from lactose. Ultrafiltration (UF) retains high-molecular-weight proteins while allowing smaller molecules like lactose, minerals, and water to pass. Protein concentration without any processing is 3.43 g/100 g. Traditional protein concentration processing method using only UF may achieve a protein concentration of 7.28 g/100 g.

As used herein, the term “diafiltration” refers to a specialized class of filtration in which the retentate is diluted with solvent and re-filtered, to reduce the concentration of soluble permeate components and increase further the concentration of retained components.

As used herein, the term “reverse osmosis” refers to the use of feed water pressure applied to a selectively permeable membrane to reduce osmotic pressure. Reverse Osmosis (RO) removes water from liquid streams, concentrating total solids but without selective separation of proteins. Traditional protein concentration processing method using only RO may achieve a protein concentration of 12.28 g/100 g.

As used herein, the term “cup” refers to a unit of volume measurement of 8 oz or 240 mL.

As used herein, the term “shelf-stable” means capable of being stored at room temperature (e.g., about 20° C. to about 25° C.) for long periods (e.g., more than 3 months) without becoming spoiled or rotten.

### EXAMPLES

The compositions and methods described herein will be more readily understood by reference to the following examples which are included merely for purposes of illustration of certain aspects and embodiments of the present disclosure, and are not intended to limit the methods and compositions described herein.

### Example 1: Exemplary Process Parameters

The process comprises two phases (Table 2). In the first phase, a diafiltration process using ultrafiltration is employed to partially concentrate the protein solution while removing unwanted low molecular weight components such as lactose. The process begins with 12 gallons of fat-free milk, which contains 128 grams of protein per gallon. The milk is processed through a 10 kDa ultrafiltration spiral membrane with a 2540 UF membrane configuration (25 inches in length, 4 inches in diameter). The system operates under a pressure of 150 psi at a temperature range of 45 to 65° F., with a 5 HP motor pump running at 50 Hz. As ultrafiltration progresses, water and small solutes permeate through the membrane, gradually reducing the solution volume to 7.5 gallons while increasing the protein concentration.

TABLE 2
Exemplary Ultrafiltration and Reverse Osmosis Parameters

| | Ultrafiltration | | Reverse Osmosis | |
|---|---|---|---|---|
| Parameter | Process | | Process | |
| Filtration Type | Ultrafiltration | | Reverse Osmosis | |
| Membrane Type | Ultrafiltration | | RO Spiral Membrane | |
| | Spiral Membrane | | | |
| Operating Pressure | 50-150 | psi | 400-600 | psi |
| Pump Frequency | 50 | Hz | 30 Hz-50 Hz | |
| Temperature Range | 40-110° | F. | 40-110° | F. |
| Membrane Element Size | 2540 UF Membrane | | 2540 RO Membrane | |
| MWCO (Molecular | 10,000 | Da | 50-80 | Da |
| Weight Cut-Off) | | | | |
| Pump Power | 5 HP Motor Pump | | 5 HP Motor Pump | |
| Initial Volume | 12 | gallons | 7.5 | gallons |
| Final Volume | 7.5 | gallons | 1.95 | gallons |
| Total Water Added | 17.4 | gallons | N/A (Water removed) | |
| Total Process Time | 5 | hours | 2 | hours |

Following ultrafiltration, diafiltration is performed to further purify the protein solution. Fresh water equivalent to 1.5 bed volumes (11.25 gallons) is introduced at the same rate of the waste water removal rate to facilitate the removal of additional small molecules. During this step, the fresh water displaces the permeate, allowing the removal of residual lactose, salts, and other small molecular-weight compounds while retaining proteins and macromolecules. The diafiltration process continues until the concentration of the 7.5-gallon ultrafiltered solution reaches the desired level for further processing (at least 50% of the lactose has been removed).

In the second phase, the reverse osmosis (RO) process is employed to further concentrate the protein solution by selectively removing water. The 7.5-gallon ultrafiltered solution is introduced into an RO filtration system equipped with a 2540 RO membrane (25 inches in length, 4 inches in diameter). The membrane has a molecular weight cut-off (MWCO) of 100-300 Da, allowing the retention of proteins while eliminating excess water. The system is operated at 600 psi using a 5 HP motor pump. The pump frequency initially starts at 50 Hz and is gradually reduced to 30 Hz as the viscosity of the solution increases, optimizing flow and efficiency.

As the reverse osmosis process continues, water is progressively removed, further concentrating the protein solution. In certain embodiments, the filtration process is maintained until the solution volume is reduced from 7.5 gallons to approximately 1.95 gallons, achieving the desired protein concentration of 50 grams per 240 mL. The entire reverse osmosis process may be completed within 2 hours, resulting in a final product with a significantly enhanced protein content and purity.

The described process may provide an efficient and scalable approach for producing high-protein concentrates suitable for nutritional, pharmaceutical, and functional food applications. The combination of ultrafiltration and reverse osmosis may ensure optimal protein retention and purification, unlike traditional filtration techniques. The adjustable pump frequency, precise membrane selection, and controlled pressure parameters may allow for improved process efficiency, reduced energy consumption, and enhanced product quality. This protein concentration process described herein may be adaptable for large-scale commercial production, providing an effective solution for the development of protein-enriched formulations.

### Example 2. Exemplary Salts in Optional Intermediate Step of Process

Between the diafiltration phase and reverse osmosis phase, salts may serve several functional roles including ionic strength adjustment, pH buffering, and protein stabilization (Table 3).

TABLE 3
Role of Salts in an Intermediate Optimization Phase

| Salt | Concentration | | Function |
|---|---|---|---|
| Calcium chloride | 10-50 | mM | Stabilizes casein micelles, |
| (CaCl₂) | | | improves heat stability |
| Sodium chloride | 10-150 | mM | Adjusts osmotic balance, |
| (NaCl) | | | modifies protein interactions |
| Potassium | 10-50 | mM | Reduces sodium content while |
| chloride (KCl) | | | maintaining ionic balance |
| Sodium citrate | 5-50 | mM | Chelates calcium, controls |
| (Na₃C₆H₅O₇) | | | protein precipitation |
| Disodium phosphate | 5-20 | mM | Buffers pH and prevents |
| (Na₂HPO₄) | | | excessive aggregation |
| Magnesium chloride | 1-10 | mM | Affects casein micelle |
| (MgCl₂) | | | structure and enzymatic activity |
| Calcium lactate | 10-30 | mM | Provides bioavailable calcium, |
| (Ca(C₃H₅O₃)₂) | | | improves texture |

Salts such as sodium chloride (NaCl) and potassium chloride (KCl) may help regulate osmotic pressure and maintain ionic balance. Sodium chloride is a standard ingredient in dairy processing, while potassium chloride is often used as a sodium-reducing alternative.

For protein stabilization and micelle structure modification, calcium chloride (CaCl₂)) is commonly added and may stabilize casein micelles and improve heat stability, though excessive use may introduce bitterness. Similarly, magnesium chloride (MgCl₂) may influence casein micelle structure and supports enzymatic activity.

To control pH and prevent protein precipitation, sodium citrate (Na₃C₆H₅O₇) may act as a chelating agent, binding calcium and ensuring protein stability. Additionally, disodium phosphate (Na₂HPO₄) may serve as a buffering agent, regulating pH and maintaining protein solubility throughout processing.

For calcium fortification and texture improvement, calcium lactate (Ca(C₃H₅O₃)₂) is often incorporated and may provide bioavailable calcium while enhancing the texture and mouthfeel of the final product.

Each of these salts is generally recognized as safe (GRAS) and used in food processing under regulatory guidelines.

### Example 3. Comparison to Existing Compositions

Table 4 highlights the differences between existing compositions and the compositions described herein.

TABLE 4
Composition of Existing Milk Products
vs. Composition described herein

| | Protein | Fat per | Protein | Fat |
|---|---|---|---|---|
| | per cup | cup | Density | Density |
| Name | (240 mL) | (240 mL) | (g/mL) | (g/mL) |
| Generic Whole Milk | 8 | 8 | 0.03 | 0.03 |
| Generic 2% Milk | 8 | 5 | 0.03 | 0.02 |
| Generic 1% Milk | 8 | 3 | 0.03 | 0.01 |
| Generic Skim Milk | 8 | 0 | 0.03 | 0.00 |
| Fairlife UF Whole Milk | 13 | 8 | 0.05 | 0.03 |
| Fairlife UF 2% Milk | 13 | 5 | 0.05 | 0.02 |
| Fairlife UF 1% Milk | 13 | 3 | 0.05 | 0.01 |
| Fairlife UF Skim Milk | 13 | 0 | 0.05 | 0.00 |
| Slate Milk | 15 | 0 | 0.06 | 0.00 |
| Fairlife Core Power Shake | 15 | 3 | 0.06 | 0.01 |
| Nestle Evaporated Milk | 16 | 16 | 0.07 | 0.07 |
| Nestle Condensed Milk | 18 | 22 | 0.08 | 0.09 |
| Fairlife Nutrition Plan Shake | 21 | 2 | 0.09 | 0.01 |
| Fairlife Core Power Elite | 24 | 2 | 0.10 | 0.01 |
| Shake | | | | |
| O-AT-KA Ultrafiltered (UF) | 30 | 1 | 0.12 | 0.00 |
| Skim | | | | |
| Composition described herein | 50+ | 0 | 0.21+ | 0.00 |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the present disclosure have been discussed, the above specification is illustrative and not restrictive. Many variations of the methods and compositions described herein will become apparent to those skilled in the art upon review of this specification and the claims below. The full scope of the present disclosure should be determined by reference to the claims, along with their full scope of equivalents, and the specification, along with such variations.

1. A liquid nutritional composition derived from milk, comprising:
protein at a protein concentration of at least about 0.15 g/mL;
optionally, fat at a fat concentration of less than about 0.01 g/mL; and
optionally, lactose, at a lactose concentration of less than about 0.01 g/mL.

2. The composition of claim 1, wherein the milk is raw milk.

3. The composition of claim 1, wherein the milk is skim milk.

4. The composition of claim 1, wherein the protein concentration is about 0.20 g/mL, about 0.25 g/mL, about 0.30 g/mL, or about 0.35 g/mL.

5-7. (canceled)

8. The composition of claim 1, wherein the protein concentration is about 0.40 g/mL.

9. The composition of claim 1, wherein the protein concentration is about 0.45 g/mL.

10. The composition of claim 1, wherein the protein concentration is about 0.50 g/mL.

11. The composition of claim 1, wherein the protein concentration is from about 0.15 g/mL to about 0.50 g/mL.

12. The composition of claim 1, wherein the fat concentration is about 0 g/mL.

13. The composition of claim 1, wherein the lactose concentration is about 0 g/mL.

14. The composition of claim 1, wherein the composition has a viscosity between about 1 mPa-s to about 5000 mPa-s.

15. The composition of claim 14, wherein the composition has viscosity of less than about 100 mPa-s.

16. The composition of claim 1, wherein the composition is a shelf-stable composition.

17. A process for preparing a liquid nutritional composition comprising:
(i) filtering a first protein solution via ultrafiltration, thereby producing a second protein solution;
(ii) filtering the second protein solution via diafiltration, thereby producing a third protein solution; and
(iii) concentrating the third protein solution via one or more water-removal techniques, thereby producing the liquid nutritional composition.

18. (canceled)

19. The process of claim 18, wherein the first protein solution is derived from skim milk or raw milk.

20-37. (canceled)

38. The process of claim 17, wherein the liquid nutritional composition comprises protein at a protein concentration of at least about 0.15 g/mL.

39-45. (canceled)

46. The process of claim 38, wherein the protein concentration is from about 0.15 g/mL to about 0.50 g/mL.

47. The process of claim 17, wherein, if the liquid nutritional composition comprises fat, the fat is present at a concentration of less than about 0.01 g/mL.

48. The process of claim 17, wherein, if the liquid nutritional composition comprises lactose, the lactose is present at a concentration of less than about 0.01 g/mL.

49. (canceled)

50. The process of claim 17, wherein the composition has viscosity of less than about 100 mPa-s.
